SEQUENCE LISTING (7 sequences)

SEQ ID NO: 1 - DNA, 29 bp, Artificial Sequence
  Description of Artificial Sequence Synthetic primer
ccgctcgagtaaccaaccaccaccaccac

SEQ ID NO: 2 - DNA, 37 bp, Artificial Sequence
  Description of Artificial Sequence Synthetic primer
ataagaatgcggccgccgttggtcctaacccttcctg

SEQ ID NO: 3 - DNA, 35 bp, Artificial Sequence
  Description of Artificial Sequence Synthetic primer
taagatgactaagccaatggggaagaattgccctg

SEQ ID NO: 4 - DNA, 33 bp, Artificial Sequence
  Description of Artificial Sequence Synthetic primer
cttccccattggcttagtcatcttattcatacc

SEQ ID NO: 5 - RNA, 8 nt, Artificial Sequence
  Description of Artificial Sequence Synthetic oligonucleotide
uaaccgaa

SEQ ID NO: 6 - DNA, 30 bp, Artificial Sequence
  Description of Artificial Sequence Synthetic oligonucleotide
gaauaagaugacuaagaauguaatggggaa

SEQ ID NO: 7 - RNA, 23 nt, Homo sapiens
aacauucauugcugucggugggu

(19) United States
(12) Patent Application Publication
(10) Pub. No.: US 2013/0017964 A1
(43) Pub. Date: Jan. 17, 2013

(54) Methods to Identify Chronic Lymphocytic Leukemia Disease Progression

(71) Applicant: Carlo M. Croce, Columbus, OH (US)

(72) Inventor: Carlo M. Croce, Columbus, OH (US)

(21) Appl. No.: 13/348,004

(22) Filed: Jan. 11, 2012

Related U.S. Application Data
(60) Provisional application No. 61/431,736, filed on Jan. 11, 2011.

(51) Int. Cl. C40B 30/04; C12Q 1/68

(52) U.S. Cl. 506/9; 435/6.12; 435/6.11

(57) ABSTRACT

The present invention provides materials and methods related to CLL disease progression. The invention provides methods related to differential expression signatures, including distinguishing histological subtypes, progression patterns, poor survival patterns, and disease-free survival patterns. Antisense and sense miRNAs, kits, and other compositions, such as pharmaceutical formulations and combination therapies are also provided.

### CROSS-REFERENCE TO RELATED APPLICATIONS

This application claims the benefit of U.S. Provisional Application No. 61/431,736 filed Jan. 11, 2011, the disclosure of which is incorporated herein by reference.

### STATEMENT REGARDING FEDERALLY SPONSORED RESEARCH

This invention was made with government support under Grant No. P01-CA81534 awarded by the National Institutes of Health. The government has certain rights in the invention.

### TECHNICAL FIELD OF THE INVENTION

The present invention is based on the observation that certain patterns of miR-181b expression correlate with certain patterns of chronic lymphocytic leukemia disease progression. Therefore, the present invention is in the fields of molecular biology and medicine.

### REFERENCE TO SEQUENCE LISTING SUBMITTED VIA EFS-WEB

This application is being filed electronically via the USPTO EFS-WEB server, as authorized and set forth in MPEP§1730 II.B.2(a)(A), and this electronic filing includes an electronically submitted sequence (SEQ ID) listing. The entire content of this sequence listing is herein incorporated by reference for all purposes. The sequence listing is identified on the electronically filed .txt file as follows: 604_{—}52615_US-SeqListing_OSURF-07026-2.txt, created on Jan. 9, 2012, and is 1,922 bytes in size.

### BACKGROUND OF THE INVENTION

Chronic lymphocytic leukemia (CLL) has a variable clinical course, varying from indolent to very aggressive. The molecular features that have been shown to be reliable for predicting an inferior clinical course in CLL are the immunoglobulin heavy-chain variable-region (IGHV) homology to the germline ≧98%, recurrent cytogenetic abnormalities and expression of zeta-associated protein (ZAP)-70 in more than ≧20% of the leukemic cells. Each of these biomarkers has been shown to be a predictor of time to treatment on univariate analysis. The clinical staging systems, based on biological and clinical parameters, are also useful for assessing prognosis in patients with CLL. The great majority of CLL are diagnosed while they are asymptomatic and in the early stages; however, these systems are not accurate enough to identify the subgroups of patients whose disease will progress. Over the past few years several studies have shown that microRNAs (miRNAs) play an important role in CLL.

### SUMMARY OF THE INVENTION

The present invention provides methods of identifying poor progression prognosis CLL status in a subject, comprising: a.) comparing the expression level of miR-181b in a first test sample from a subject with CLL and at least one successive test samples from a subject with CLL, b.) identifying poor progression prognosis CLL status in a subject if miR-181b level is decreased from first test sample the at least one successive test sample, or c.) identifying no poor progression prognosis CLL status in a subject if miR-181b level is not decreased from the first test sample the at least one successive test sample.

The present invention also provides methods of identifying poor progression prognosis CLL status in a subject, comprising: a.) determining the expression level of miR-181b in at least one first test sample; b.) determining the expression level of miR-181b in at least one test sample successive to the first test sample; c.) identifying poor progression prognosis CLL status in a subject if the miR181b level as determined in step b.) is less than miR-181b level as determined in step a.)

Also provided are such methods as described, wherein poor progression prognosis CLL status is identified if the miR-181b level as determined in step b.) is at least 20% less than miR-181b level as determined in step a.)

Also provided are such methods as described, wherein poor progression prognosis CLL status is identified if the miR-181b level as determined in step b.) is at least 30% less than miR-181b level as determined in step a.).

Also provided are such methods as described, wherein poor progression prognosis CLL status is identified if the miR-181b level as determined in step b.) is at least 40% less than miR-181b level as determined in step a.).

Also provided are such methods as described, wherein poor progression prognosis CLL status is identified if the miR-181b level as determined in step b.) is at least 50% less than miR-181b level as determined in step a.).

Also provided are such methods as described, wherein poor progression prognosis CLL status is identified if the miR-181b level as determined in step b.) is at least 60% less than miR-181b level as determined in step a.).

Also provided are such methods as described, wherein step b.) is at least six months after step a.).

Also provided are such methods as described, wherein step b.) is at least twelve months after step a.).

Also provided are such methods as described, wherein step b.) is at least eighteen months after step a.).

Also provided are such methods as described, wherein step b.) is at least twenty-four months after step a.).

Also provided are such methods as described, which further comprises identifying clinical stage if poor progression prognosis CLL status is identified.

Also provided are such methods as described, which further comprises identifying need for treatment if poor progression prognosis CLL status is identified.

Also provided are such methods as described, which further comprises identifying aggressive form of CLL if poor prognosis CLL status is identified.

Also provided are such methods as described, wherein a level of expression of miR-181b is assessed by detecting the presence of a transcribed polynucleotide or portion thereof, wherein the transcribed polynucleotide comprises a coding region of miR-181b gene product.

Also provided are such methods as described, wherein steps a. and b. are performed in vitro.

Also provided are such methods as described, wherein the sample is a CLL-associated body fluid or tissue.

Also provided are such methods as described, wherein the sample comprises cells obtained from the patient.

The present invention also provides methods of identifying poor progression prognosis CLL status in a subject, comprising: a.) comparing the expression level of at least one miR selected from the group consisting of: miR-130b; miR126; miR-296-3p; and miR-223 in a first test sample from a subject with CLL and at least one successive test samples from a subject with CLL, b.) identifying poor progression prognosis CLL status in a subject if the at least one miR expression level is decreased from first test sample the at least one successive test sample, or c.) identifying no poor progression prognosis CLL status in a subject if the miR expression level is not decreased from the first test sample the at least one successive test sample.

The present invention also provides methods of identifying poor progression prognosis CLL status in a subject, comprising: a.) determining the expression level of at least one miR selected from the group consisting of: miR-130b; miR126; miR-296-3p; and miR-223 in at least one first test sample; b.) determining the expression level of at least one miR selected from the group consisting of: miR-130b; miR126; miR-296-3p; and miR-223 in at least one test sample successive to the first test sample; c.) identifying poor progression prognosis CLL status in a subject if the at least one miR expression level as determined in step b.) is less than the at least one miR expression level as determined in step a.)

Also provided are such methods as described, wherein poor progression prognosis CLL status is identified if the miR expression level as determined in step b.) is at least 50% less than miR expression level as determined in step a.).

Also provided are such methods as described, wherein step b.) is at least one year after step a.)

In addition, provided herein are compositions of matter, comprising sense miR-181b and an anti-CLL therapeutic agents, or a pharmaceutically-acceptable formulations thereof.

Also provided are such compositions, wherein the anti-CLL therapeutic agent is selected from the group consisting of: cyclophosphamide, vincristine, and prednisone. Other drug choices include fludarabine, chlorambucil, hydroxyurea (hydroxycarbamide), cytarabine, busulfan, rituximab, alemtuzumab, Allopurinol, Imatinib (Gleevec) Nilotinib (Tasigna), Immune globulin (IG), and Bendamustine hydrochloride (Treanda).

Also provided are kits comprising such compositions.

Also provided are methods to affect at least one human CLL cell, comprising introducing at least one sense miR-181b and at least one anti-CLL therapeutic agent to at least one human CLL cell.

Also provided are such methods wherein the anti-CLL therapeutic agent is selected from the group consisting of: cyclophosphamide, vincristine, and prednisone. Other drug choices include fludarabine, chlorambucil, hydroxyurea (hydroxycarbamide), cytarabine, busulfan, rituximab, alemtuzumab, Allopurinol, Imatinib (Gleevec) Nilotinib (Tasigna), Immune globulin (IG), and Bendamustine hydrochloride (Treanda).

Also provided are such methods wherein the at least one human CLL cell is present in vitro.

Also provided are such methods wherein the at least one human CLL cell is present in situ.

Also provided are such methods wherein the at least one human CLL cell is present in vivo.

Also provided are such methods which results in reduction in progression of CLL disease.

Also provided are methods to treat a patient with CLL, comprising: a.) identifying if a patient with CLL has decreased miR-181b expression compared to control, b.) treating the patient with sense miR-181b if the patient has decreased miR-181b expression compared to control.

Also provided are methods to identify useful compounds, comprising a.) introducing a test compound and antisense miR-181b to CLL cells, and b.) identifying test compounds useful to affect CLL cells.

Also provided are methods to identify cancer cell sample status, comprising: a.) correlating miR-181b status in a cell test sample with control, and b.) identifying cancer cell sample status.

Also provided are methods to predict CLL cancer cell sample status, comprising: a.) correlating miR-181b status in a CLL cell-containing test sample with control, and b.) predicting CLL cell sample status.

Also provided are such methods to identify organism CLL status, comprising: a.) correlating miR-181b status in a organism-derived test sample with control, and b.) identifying organism status.

### BRIEF DESCRIPTION OF THE FIGURES

The application contains one or more figures executed in color and/or one or more photographs. Copies of color figures(s) and/or photograph(s) will be provided upon request and payment of the necessary fee.

FIGS. 1A-1B. MiR-181b expression values significantly decrease in progressive but not in stable CLL over time (training set). Relative expression of the mature miR-181b, in the first time point (violet blocks) and last time point (yellow blocks) from sequential samples of CLL patients with either progressive (FIG. 1A) or stable (FIG. 1B) disease. The expression has been determined by stem-loop qRT-PCR. Each sample data was normalized to the endogenous reference RNU44 by using 2^{-Δct} method. P value is the result of the paired ttest on Log₁₀ transformed values.

FIGS. 2A-2D. MiR-181b expression values discriminate stable from progressive CLLs. Relative expression of the mature miR-181b in serial time points from CLL patients with a progressive (violet dots) or stable (yellow dots) disease in the training (FIG. 2A) and validation (FIG. 2B) set. The bars indicate the mean values and the errors. P value is the result of the Mann-Whitney test. Patients with (violet lines) or without (yellow lines) the properties, defined on the base of miR-181b expression values are indicated in training (FIG. 2C) or validation (FIG. 2D) set. The ending points are showed with either circle for patients with IGHV≦98%, or cross for those with IGHV≧98% and with square for unknown values. P value is the result of the Fisher's exact test. MiR-181b relative expression is determined by stem-loop qRT-PCR. Each sample data was normalized to the endogenous reference RNU44 using 2^{-Δct} method.

FIGS. 3A-3C. Relationship between miR-181b expression values and time to treatment. Kaplan-Maier curves depict the clinical outcome of CLL patients in which the two groups were separated on the base of either a miRNA value dichotomized to “≦0.005” (FIG. 3A and FIG. 3B) or the properties defined of the base of miR-181 expression values (FIG. 3C). The patient numbers were measured at the time intervals of 0, 500, 1000, 1500, 2000, and 2500 days. Log-rank P values are from Kaplan-Meier analysis.

FIGS. 4A-4D. MiR-181b targets CLL genes related. (FIG. 4A) Putative binding site of miR-181b in MCL1 3′UTRs (TargetScan5.1 Database) showing, in order [SEQ ID NOs: 5, 6, 7]. Asterisks indicate the nucleotides substituted in miR-181b predicted target site to perform luciferase assay. (FIG. 4B) MCL1 3′UTRs regulated luciferase activity dependent on miR-181b in HeLa cell lines (WT, wild-type; MUT, mutant; P value, t-test). Firefly luciferase activity was normalized on Renilla luciferase activity of the gene included in the same vector. (FIG. 4C) Western blot analysis of MCL1 after either scrambled sequence or precursor-miR-181b transfection in HeLa cell line, β-actin has been used as loading control. Cells were collected after 48 and 72hrs of miRNA transfection. (FIG. 4D), Densitometric display of the TCL1, MCL1 and BCL2 western blot analysis (FIG. 6) normalized on β-actin on samples from CLL patients with either progressive or stable disease. Asterisks indicate patients in which the protein expression of the targets increase. R, correlation factor, Pearson's test. In. time point, intermediate time point.

FIGS. 5A-5B. MiR-181b expression values significantly decreases in progressive but not in stable CLL over time (validation set). Relative expression of the mature miR-181b, in the first time point (violet blocks) and last time point (yellow blocks) from sequential samples of CLL patients with a progressive (FIG. 5A) or stable (FIG. 5B) disease. The expression has been determined by stem-loop qRT-PCR. Each sample data was normalized to the endogenous reference RNU44 by using 2̂-^{Δct} method. P value is the result of the paired t test on log₁₀ transformed values.

FIG. 6. TCL1, MCL1 and BCL2 protein expression in patients with either progressive or stable disease. Western Blot analysis of TCL1, MCL1 and BCL2 proteins on leukemic cells from peripheral blood of patients with either progressive or stable disease. β-actin has been used as loading control. Densitometric analysis is shown in FIG. 4.

### DETAILED DESCRIPTION OF THE INVENTION

MicroRNAs play a crucial role in Chronic Lymphocytic Leukemia (CLL). The inventors investigated whether microRNAs can discriminate patients with a progressive disease from those with a stable disease.

The inventors determined indicators of disease progression in CLL by performing a genome-wide miRNA profiling on sequential samples obtained from patients presenting at different clinical stages. The inventors then validated the result using samples obtained from patients with a stable disease and in an independent larger cohort of sequential samples from patients with either progressive or stable disease. Protein expression of critical targets of the dysregulated miRNA was also analyzed in a representative set of samples

The inventors analyzed 358 sequential samples from 114 patients. MicroRNA expression profiling was performed on the leukemic cells isolated from each sequential samples obtained from patients with progressive disease. The expression of the most dysregulated microRNA was examined in the samples obtained from CLL patients with a stable disease and validated in an independent larger cohort.

Parameters discriminating progressive from stable disease, based on the microRNA values, were associated with time to treatment. Gene targets of the selected microRNA were analyzed for their protein expression in a representative set of samples. During the course of the disease the expression values of miR-181b decreased in samples from patients with a progressive (P<0.001, training and validation sets) but not in those from patients with a stable disease (P=0.3, training set; P=0.2, validation set) over time.

A drop of 50% or more between sequential samples and/or a miR-181b value ≦0.005 at the starting time point were significant to differentiate progressive from stable disease (P=0.004, training set; P<0.001, validation set). These parameters were associated with high risk of treatment (risk ratio, 5.8; 95% confidence interval, 2.5-14.9). Decrease of miR-181b inversely correlated with increased expression of anti-apoptotic target genes. Parameters defined on the basis of the miR-181b expression values specify disease progression in CLL and are associated with clinical outcome.

The inventors determined whether miRNAs play a role in CLL progression. The inventors evaluated two independent cohorts of patients over time and found that miR-181b expression values significantly decreased in samples obtained from patients with progressive disease but not in samples obtained from patients with stable disease. The inventors also observed that several CLL patients, mostly with progressive disease, had miR-181b expression values ≦0.005 at the starting time point.

The inventors measured the expression of miR-181b in 16 samples of B-CD19+ and B-CD19+/CD5+ cells obtained from healthy individuals. Neither of these control samples had such low miR-181b expression (data not shown). The miR-181b value ≦0.005 was chosen as low in this study because it was observed in very few of the samples obtained from CLL patients with stable disease in the training set and this enabled the inventors to discriminate, at least in part, the two manifestations of the disease.

Therefore, considering a decrease in the miR-181b expression values of 50% or more between sequential samples and/or miR-181b value ≦0.005 at the starting point, the inventors observed that the proportion of progressive patients that have these properties was significantly higher than the proportion for those with stable disease, indicating that the miR-181b is a biomarker for the disease progression in CLL. To further corroborate the findings, the inventors demonstrated a strong correlation between the low expression of miR-181b at the last time point and the risk of requiring treatment in both the training and the validation cohorts; which is consistent with the previous study on CLL with 17p deletion.

Last time point was chosen for this analysis since the inventors have shown a major difference at the ending than at the starting time points between the 2 subgroups with either progressive or stable disease. However, the drop of 50% occurs in many progressive cases, prior to the detection of the classical clinical parameters, which are represented by the last time point (Table 3). To date, the prediction of the risk of need of treatment based only on miR-181b expression values is not reliable in the laboratory practice.

To address this point, the inventors correlated samples with the properties previously defined and time to first treatment in a Cox regression analysis. The inventors found that these patients have a risk of requiring treatment that is 5.8 times that of patients without this condition. This was the case in both validation and training sets, even though in the test cohort the result was not significant because of the small number of samples and 5 samples diagnosed as progressive and having the property were untreated at the last follow-up.

MiR-181b targets TCL1, whose enhanced expression in murine models leads to the aggressive form of CLL, and the anti-apoptotic genes BCL2 and MCL1 that are up-modulated in most aggressive CLLs.

The inventors have now found that while the MCL1 and BCL2 expression increased in samples from patients with progressive disease but not in samples from patients with stable disease over time, TCL1 increased in both sets, indicating a predominant role for this gene in the early phase of CLL. Overall, in 14 out of 18 patients with a progressive disease, at least 1 of the 3 targets increased during disease progression, suggesting a functional role of this miRNA.

MiR-181b also targets activation-induced cytidine deaminase (AID). Thus, loss of miR-181b will lead to overexpression of AID, leading to genomic instability and cancer progression.

MiR-181b is a unique biomarker for CLL disease progression since its expression can be monitored throughout the disease course of a patient and this change in the leukemic cells correlate with the overexpression of 4 genes with great significance in CLL and other cancer (i.e., MCL1, TCL1, BCL2 and AID). Collectively, this information together with the analysis of stable prognostic markers (i.e. ZAP-70 and IGHV mutations status) would enable a physician to make a more accurate decision on the treatment strategy. Moreover, since MiR-181b maps at 1q31.3 and 9q33.3, each or both precursor 181(b) molecules may be used for research, assays and/or therapy, as discussed herein.

To date, although treatment of early-stage patients finds no survival advantage and “wait and watch” is the standard care for CLL patients, the development of more active therapies have rekindled interest in studying the benefit of early treatment for selected high-risk patients with CLL; hence in this new context the identification of biomarkers predicting disease progression is extremely useful.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The present invention provides research tools, diagnostic methods, and therapeutical methods and compositions using the knowledge derived from this discovery. The invention is industrially applicable for the purpose of sensitizing tumor cells to drug-inducing apoptosis and also to inhibit tumor cell survival, proliferation and invasive capabilities.

### Definitions and Abbreviations

DNA Deoxyribonucleic acid

mRNA Messenger RNA

PCR Polymerase chain reaction

pre-miRNA Precursor microRNA

qRT-PCR Quantitative reverse transcriptase polymerase chain reaction

RNA Ribonucleic acid

It is to be understood that both the foregoing general description and the following detailed description are exemplary and explanatory only and are not intended to limit the scope of the current teachings. In this application, the use of the singular includes the plural unless specifically stated otherwise.

The use of the word “a” or “an” when used in conjunction with the term “comprising” in the claims and/or the specification may mean “one,” but it is also consistent with the meaning of “one or more,” “at least one,” and “one or more than one.”

Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

It is understood that an miRNA is derived from genomic sequences or a gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary miRNA probes of the invention can be or be at least 60, 65, 70, 75, 80, 85, 90, 95, or 100% complementary to their target.

The term “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adjunctive therapy: A treatment used in combination with a primary treatment to improve the effects of the primary treatment.

Clinical outcome: Refers to the health status of a patient following treatment for a disease or disorder or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Control: A “control” refers to a sample or standard used for comparison with an experimental sample, such as a tumor sample obtained from a patient.

Decrease in survival: As used herein, “decrease in survival” refers to a decrease in the length of time before death of a patient, or an increase in the risk of death for the patient.

Detecting level of expression: For example, “detecting the level of miR or miRNA expression” refers to quantifying the amount of miR or miRNA present in a sample. Detecting expression of the specific miR, or any microRNA, can be achieved using any method known in the art or described herein, such as by qRT-PCR. Detecting expression of miR includes detecting expression of either a mature form of miRNA or a precursor form that is correlated with miRNA expression. Typically, miRNA detection methods involve sequence specific detection, such as by RT-PCR. miR-specific primers and probes can be designed using the precursor and mature miR nucleic acid sequences, which are known in the art and provided herein as in the SEQ ID NOs.

MicroRNA (miRNA): Single-stranded RNA molecules that regulate gene expression. MicroRNAs are generally 21-23 nucleotides in length. MicroRNAs are processed from primary transcripts known as pri-miRNA to short stem-loop structures called precursor (pre)-miRNA and finally to functional, mature microRNA. Mature microRNA molecules are partially-complementary to one or more messenger RNA molecules, and their primary function is to down-regulate gene expression. MicroRNAs regulate gene expression through the RNAi pathway.

miR expression: As used herein, “low miR expression” and “high miR expression” are relative terms that refer to the level of miRNAs found in a sample. In some embodiments, low and high miR expression is determined by comparison of miRNA levels in a group of control samples and test samples. Low and high expression can then be assigned to each sample based on whether the expression of mi in a sample is above (high) or below (low) the average or median miR expression level. For individual samples, high or low miR expression can be determined by comparison of the sample to a control or reference sample known to have high or low expression, or by comparison to a standard value. Low and high miR expression can include expression of either the precursor or mature forms of miRNA, or both.

Patient: As used herein, the term “patient” includes human and non-human animals. The preferred patient for treatment is a human. “Patient” and “subject” are used interchangeably herein.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Screening: As used herein, “screening” refers to the process used to evaluate and identify candidate agents that affect such disease. Expression of a microRNA can be quantified using any one of a number of techniques known in the art and described herein, such as by microarray analysis or by qRT-PCR.

Small molecule: A molecule, typically with a molecular weight less than about 1000 Daltons, or in some embodiments, less than about 500 Daltons, wherein the molecule is capable of modulating, to some measurable extent, an activity of a target molecule.

Therapeutic: A generic term that includes both diagnosis and treatment.

Therapeutic agent: A chemical compound, small molecule, or other composition, such as an antisense compound, antibody, protease inhibitor, hormone, chemokine or cytokine, capable of inducing a desired therapeutic or prophylactic effect when properly administered to a subject.

As used herein, a “candidate agent” is a compound selected for screening to determine if it can function as a therapeutic agent. “Incubating” includes a sufficient amount of time for an agent to interact with a cell or tissue. “Contacting” includes incubating an agent in solid or in liquid form with a cell or tissue. “Treating” a cell or tissue with an agent includes contacting or incubating the agent with the cell or tissue.

Therapeutically-effective amount: A quantity of a specified pharmaceutical or therapeutic agent sufficient to achieve a desired effect in a subject, or in a cell, being treated with the agent. The effective amount of the agent will be dependent on several factors, including, but not limited to the subject or cells being treated, and the manner of administration of the therapeutic composition.

In some embodiments of the present methods, use of a control is desirable. In that regard, the control may be a non-cancerous tissue sample obtained from the same patient, or a tissue sample obtained from a healthy subject, such as a healthy tissue donor. In another example, the control is a standard calculated from historical values. Tumor samples and non-cancerous tissue samples can be obtained according to any method known in the art. For example, tumor and non-cancerous samples can be obtained from cancer patients that have undergone resection, or they can be obtained by extraction using a hypodermic needle, by microdissection, or by laser capture. Control (non-cancerous) samples can be obtained, for example, from a cadaveric donor or from a healthy donor.

In some embodiments, screening comprises contacting the candidate agents with cells. The cells can be primary cells obtained from a patient, or the cells can be immortalized or transformed cells.

The candidate agents can be any type of agent, such as a protein, peptide, small molecule, antibody or nucleic acid. In some embodiments, the candidate agent is a cytokine. In some embodiments, the candidate agent is a small molecule. Screening includes both high-throughout screening and screening individual or small groups of candidate agents.

MicroRNA detection: In some methods herein, it is desirable to identify miRNAs present in a sample.

### General Description

The sequences of precursor microRNAs (pre-miRNAs) and mature miRNAs are publicly available, such as through the miRBase database, available online by the Sanger Institute (see Griffiths-Jones et al., Nucleic Acids Res. 36:D154-D158, 2008; Griffiths-Jones et al., Nucleic Acids Res. 34:D140-D144, 2006; and Griffiths-Jones, Nucleic Acids Res. 32:D109-D111, 2004). The sequences of the precursor and mature forms of the presently disclosed preferred family members are provided herein.

Detection and quantification of RNA expression can be achieved by any one of a number of methods well known in the art (see, for example, U.S. Patent Application Publication Nos. 2006/0211000 and 2007/0299030, herein incorporated by reference) and described below. Using the known sequences for RNA family members, specific probes and primers can be designed for use in the detection methods described below as appropriate.

In some cases, the RNA detection method requires isolation of nucleic acid from a sample, such as a cell or tissue sample. Nucleic acids, including RNA and specifically miRNA, can be isolated using any suitable technique known in the art. For example, phenol-based extraction is a common method for isolation of RNA. Phenol-based reagents contain a combination of denaturants and RNase inhibitors for cell and tissue disruption and subsequent separation of RNA from contaminants. Phenol-based isolation procedures can recover RNA species in the 10-200-nucleotide range (e.g., precursor and mature miRNAs, 5S and 5.8S ribosomal RNA (rRNA), and U1 small nuclear RNA (snRNA)). In addition, extraction procedures such as those using TRIZOLTM or TRI REAGENTTM, will purify all RNAs, large and small, and are efficient methods for isolating total RNA from biological samples that contain miRNAs and small interfering RNAs (siRNAs).

In some embodiments, use of a microarray is desirable. A microarray is a microscopic, ordered array of nucleic acids, proteins, small molecules, cells or other substances that enables parallel analysis of complex biochemical samples. A DNA microarray consists of different nucleic acid probes, known as capture probes that are chemically attached to a solid substrate, which can be a microchip, a glass slide or a microsphere-sized bead. Microarrays can be used, for example, to measure the expression levels of large numbers of messenger RNAs (mRNAs) and/or miRNAs simultaneously.

Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays.

Microarray analysis of miRNAs, for example (although these procedures can be used in modified form for any RNA analysis) can be accomplished according to any method known in the art (see, for example, PCT Publication No. WO 2008/054828; Ye et al., Nat. Med. 9(4):416-423, 2003; Calin et al., N. Engl. J. Med. 353(17):1793-1801, 2005, each of which is herein incorporated by reference). In one example, RNA is extracted from a cell or tissue sample, the small RNAs (18-26-nucleotide RNAs) are size-selected from total RNA using denaturing polyacrylamide gel electrophoresis. Oligonucleotide linkers are attached to the 5′ and 3′ ends of the small RNAs and the resulting ligation products are used as templates for an RT-PCR reaction with 10 cycles of amplification. The sense strand PCR primer has a fluorophore attached to its 5′ end, thereby fluorescently labeling the sense strand of the PCR product. The PCR product is denatured and then hybridized to the microarray. A PCR product, referred to as the target nucleic acid that is complementary to the corresponding miRNA capture probe sequence on the array will hybridize, via base pairing, to the spot at which the capture probes are affixed. The spot will then fluoresce when excited using a microarray laser scanner. The fluorescence intensity of each spot is then evaluated in terms of the number of copies of a particular miRNA, using a number of positive and negative controls and array data normalization methods, which will result in assessment of the level of expression of a particular miRNA.

In an alternative method, total RNA containing the small RNA fraction (including the miRNA) extracted from a cell or tissue sample is used directly without size-selection of small RNAs, and 3′ end labeled using T4 RNA ligase and either a fluorescently-labeled short RNA linker The RNA samples are labeled by incubation at 30° C. for 2 hours followed by heat inactivation of the T4 RNA ligase at 80° C. for 5 minutes. The fluorophore-labeled miRNAs complementary to the corresponding miRNA capture probe sequences on the array will hybridize, via base pairing, to the spot at which the capture probes are affixed. The microarray scanning and data processing is carried out as described above.

There are several types of microarrays than be employed, including spotted oligonucleotide microarrays, pre-fabricated oligonucleotide microarrays and spotted long oligonucleotide arrays. In spotted oligonucleotide microarrays, the capture probes are oligonucleotides complementary to miRNA sequences. This type of array is typically hybridized with amplified PCR products of size-selected small RNAs from two samples to be compared (such as non-cancerous tissue and cancerous or sample tissue) that are labeled with two different fluorophores. Alternatively, total RNA containing the small RNA fraction (including the miRNAs) is extracted from the two samples and used directly without size-selection of small RNAs, and 3′ end labeled using T4 RNA ligase and short RNA linkers labeled with two different fluorophores. The samples can be mixed and hybridized to one single microarray that is then scanned, allowing the visualization of up-regulated and down-regulated miRNA genes in one assay.

In pre-fabricated oligonucleotide microarrays or single-channel microarrays, the probes are designed to match the sequences of known or predicted miRNAs. There are commercially available designs that cover complete genomes (for example, from Affymetrix or Agilent). These microarrays give estimations of the absolute value of gene expression and therefore the comparison of two conditions requires the use of two separate microarrays.

Spotted long oligonucleotide arrays are composed of 50 to 70-mer oligonucleotide capture probes, and are produced by either ink-jet or robotic printing. Short Oligonucleotide Arrays are composed of 20-25-mer oligonucleotide probes, and are produced by photolithographic synthesis (Affymetrix) or by robotic printing.

In some embodiments, use of quantitative RT-PCR is desirable. Quantitative RT-PCR (qRT-PCR) is a modification of polymerase chain reaction used to rapidly measure the quantity of a product of polymerase chain reaction. qRT-PCR is commonly used for the purpose of determining whether a genetic sequence, such as a miR, is present in a sample, and if it is present, the number of copies in the sample. Any method of PCR that can determine the expression of a nucleic acid molecule, including a miRNA, falls within the scope of the present disclosure. There are several variations of the qRT-PCR method known in the art, three of which are described below.

Methods for quantitative polymerase chain reaction include, but are not limited to, via agarose gel electrophoresis, the use of SYBR Green (a double stranded DNA dye), and the use of a fluorescent reporter probe. The latter two can be analyzed in real-time.

With agarose gel electrophoresis, the unknown sample and a known sample are prepared with a known concentration of a similarly sized section of target DNA for amplification. Both reactions are run for the same length of time in identical conditions (preferably using the same primers, or at least primers of similar annealing temperatures). Agarose gel electrophoresis is used to separate the products of the reaction from their original DNA and spare primers. The relative quantities of the known and unknown samples are measured to determine the quantity of the unknown.

The use of SYBR Green dye is more accurate than the agarose gel method, and can give results in real time. A DNA binding dye binds all newly synthesized double stranded DNA and an increase in fluorescence intensity is measured, thus allowing initial concentrations to be determined. However, SYBR Green will label all double-stranded DNA, including any unexpected PCR products as well as primer dimers, leading to potential complications and artifacts. The reaction is prepared as usual, with the addition of fluorescent double-stranded DNA dye. The reaction is run, and the levels of fluorescence are monitored (the dye only fluoresces when bound to the double-stranded DNA). With reference to a standard sample or a standard curve, the double-stranded DNA concentration in the PCR can be determined

The fluorescent reporter probe method uses a sequence-specific nucleic acid based probe so as to only quantify the probe sequence and not all double stranded DNA. It is commonly carried out with DNA based probes with a fluorescent reporter and a quencher held in adjacent positions (so-called dual-labeled probes). The close proximity of the reporter to the quencher prevents its fluorescence; it is only on the breakdown of the probe that the fluorescence is detected. This process depends on the 5′ to 3′ exonuclease activity of the polymerase involved.

The real-time quantitative PCR reaction is prepared with the addition of the dual-labeled probe. On denaturation of the double-stranded DNA template, the probe is able to bind to its complementary sequence in the region of interest of the template DNA. When the PCR reaction mixture is heated to activate the polymerase, the polymerase starts synthesizing the complementary strand to the primed single stranded template DNA. As the polymerization continues, it reaches the probe bound to its complementary sequence, which is then hydrolyzed due to the 5′-3′ exonuclease activity of the polymerase, thereby separating the fluorescent reporter and the quencher molecules. This results in an increase in fluorescence, which is detected. During thermal cycling of the real-time PCR reaction, the increase in fluorescence, as released from the hydrolyzed dual-labeled probe in each PCR cycle is monitored, which allows accurate determination of the final, and so initial, quantities of DNA.

In some embodiments, use of in situ hybridization is desirable. In situ hybridization (ISH) applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of miRNAs.

Sample cells or tissues are treated to increase their permeability to allow a probe, such as a miRNA-specific probe, to enter the cells. The probe is added to the treated cells, allowed to hybridize at pertinent temperature, and excess probe is washed away. A complementary probe is labeled with a radioactive, fluorescent or antigenic tag, so that the probe's location and quantity in the tissue can be determined using autoradiography, fluorescence microscopy or immunoassay. The sample may be any sample as herein described, such as a non-cancerous or cancerous tissue sample. Since the sequences of miR family members are known, miR probes can be designed accordingly such that the probes specifically bind miR.

In some embodiments, use of in situ PCR is desirable. In situ PCR is the PCR based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products is generally achieved by one of two different techniques, indirect in situ PCR by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

Use of differentially-expressed miRs and miRNAs as predictive markers of prognosis and for identification of therapeutic agents. It is disclosed herein that certain expression patterns of miRs along with status indicators are predictors of survival prognosis in certain patients. As used herein, “poor prognosis” generally refers to a decrease in survival, or in other words, an increase in risk of death or a decrease in the time until death. Poor prognosis can also refer to an increase in severity of the disease, such as an increase in spread (metastasis) of the cancer to other organs. In one embodiment, the respective markers show at least a 1.5-fold increase or decrease in expression relative to the control. In other embodiments, poor prognosis is indicated by at least a 2-fold, at least a 2.5-fold, at least a 3-fold, at least a 3.5-fold, or at least a 4-fold increase or decrease in the markers relative to the wild-type tumor control figures.

Methods of screening candidate agents to identify therapeutic agents for the treatment of disease are well known in the art. Methods of detecting expression levels of RNA and proteins are known in the art and are described herein, such as, but not limited to, microarray analysis, RT-PCR (including qRT-PCR), in situ hybridization, in situ PCR, and Northern blot analysis. In one embodiment, screening comprises a high-throughput screen. In another embodiment, candidate agents are screened individually.

The candidate agents can be any type of molecule, such as, but not limited to nucleic acid molecules, proteins, peptides, antibodies, lipids, small molecules, chemicals, cytokines, chemokines, hormones, or any other type of molecule that may alter cancer disease state(s) either directly or indirectly.

Typically, an endogenous gene, miRNA or mRNA is modulated in the cell. In particular embodiments, the nucleic acid sequence comprises at least one segment that is at least 70, 75, 80, 85, 90, 95, or 100% identical in nucleic acid sequence to one or more miRNA sequence listed in Table 1. Modulation of the expression or processing of an endogenous gene, miRNA, or mRNA can be through modulation of the processing of a mRNA, such processing including transcription, transportation and/or translation with in a cell. Modulation may also be effected by the inhibition or enhancement of miRNA activity with a cell, tissue, or organ. Such processing may effect the expression of an encoded product or the stability of the mRNA. In still other embodiments, a nucleic acid sequence can comprise a modified nucleic acid sequence. In certain aspects, one or more miRNA sequence may include or comprise a modified nucleobase or nucleic acid sequence.

It will be understood in methods of the invention that a cell or other biological matter such as an organism (including patients) can be provided an miRNA or miRNA molecule corresponding to a particular miRNA by administering to the cell or organism a nucleic acid molecule that functions as the corresponding miRNA once inside the cell. The form of the molecule provided to the cell may not be the form that acts a miRNA once inside the cell. Thus, it is contemplated that in some embodiments, biological matter is provided a synthetic miRNA or a nonsynthetic miRNA, such as one that becomes processed into a mature and active miRNA once it has access to the cell's miRNA processing machinery. In certain embodiments, it is specifically contemplated that the miRNA molecule provided to the biological matter is not a mature miRNA molecule but a nucleic acid molecule that can be processed into the mature miRNA once it is accessible to miRNA processing machinery. The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa. It will be understand that the term “providing” an agent is used to include “administering” the agent to a patient.

In certain embodiments, methods also include targeting a miRNA to modulate in a cell or organism. The term “targeting a miRNA to modulate” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with a miRNA inhibitor, which effectively inhibits the targeted miRNA in the cell or organism (negative modulation).

In some embodiments, the miRNA targeted to be modulated is a miRNA that affects a disease, condition, or pathway. In certain embodiments, the miRNA is targeted because a treatment can be provided by negative modulation of the targeted miRNA. In other embodiments, the miRNA is targeted because a treatment can be provided by positive modulation of the targeted miRNA.

In certain methods of the invention, there is a further step of administering the selected miRNA modulator to a cell, tissue, organ, or organism (collectively “biological matter”) in need of treatment related to modulation of the targeted miRNA or in need of the physiological or biological results discussed herein (such as with respect to a particular cellular pathway or result like decrease in cell viability). Consequently, in some methods of the invention there is a step of identifying a patient in need of treatment that can be provided by the miRNA modulator(s). It is contemplated that an effective amount of a miRNA modulator can be administered in some embodiments. In particular embodiments, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a symptom. For example, with respect to cancer, it is contemplated that a therapeutic benefit can be inhibition of tumor growth, prevention of metastasis, reduction in number of metastases, inhibition of cancer cell proliferation, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, and/or delay of death directly or indirectly related to cancer.

Furthermore, it is contemplated that the miRNA compositions may be provided as part of a therapy to a patient, in conjunction with traditional therapies or preventative agents. Moreover, it is contemplated that any method discussed in the context of therapy may be applied as preventatively, particularly in a patient identified to be potentially in need of the therapy or at risk of the condition or disease for which a therapy is needed.

In addition, methods of the invention concern employing one or more nucleic acids corresponding to a miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating cancer in a patient comprising administering to the patient the cancer therapeutic and an effective amount of at least one miRNA molecule that improves the efficacy of the cancer therapeutic or protects non-cancer cells. Cancer therapies also include a variety of combination therapies with both chemical and radiation based treatments. Combination chemotherapies include but are not limited to, for example, bevacizumab, cisplatin (CDDP), carboplatin, EGFR inhibitors (gefitinib and cetuximab), procarbazine, mechlorethamine, cyclophosphamide, camptothecin, COX-2 inhibitors (e.g., celecoxib) ifosfamide, melphalan, chlorambucil, busulfan, nitrosurea, dactinomycin, daunorubicin, doxorubicin (adriamycin), bleomycin, plicomycin, mitomycin, etoposide (VP16), tamoxifen, raloxifene, estrogen receptor binding agents, taxol, taxotere, gemcitabien, navelbine, farnesyl-protein transferase inhibitors, transplatinum, 5-fluorthe ouracil, vincristin, vinblastin and methotrexate, or any analog or derivative variant of the foregoing.

Generally, inhibitors of miRNAs can be given to achieve the opposite effect as compared to when nucleic acid molecules corresponding to the mature miRNA are given Similarly, nucleic acid molecules corresponding to the mature miRNA can be given to achieve the opposite effect as compared to when inhibitors of the miRNA are given. For example, miRNA molecules that increase cell proliferation can be provided to cells to increase proliferation or inhibitors of such molecules can be provided to cells to decrease cell proliferation. The present invention contemplates these embodiments in the context of the different physiological effects observed with the different miRNA molecules and miRNA inhibitors disclosed herein. These include, but are not limited to, the following physiological effects: increase and decreasing cell proliferation, increasing or decreasing apoptosis, increasing transformation, increasing or decreasing cell viability, reduce or increase viable cell number, and increase or decrease number of cells at a particular phase of the cell cycle. Methods of the invention are generally contemplated to include providing or introducing one or more different nucleic acid molecules corresponding to one or more different miRNA molecules. It is contemplated that the following, at least the following, or at most the following number of different nucleic acid molecules may be provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein. This also applies to the number of different miRNA molecules that can be provided or introduced into a cell.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

The following examples are provided to illustrate certain particular features and/or embodiments. These examples should not be construed to limit the disclosure to the particular features or embodiments described.

### EXAMPLES

### Example 1

### Synthesis of the LLL12 and Related Compounds

Patients and Analysis of ZAP-70 and IGHV Mutation Status

The inventors selected 358 samples obtained from 114 untreated patients who were diagnosed for CLL and were enrolled in the CLL Research Consortium upon written informed consent. The participating CRC institutions provided the clinical data associated with each of the times points of the patients, including the date of the initiation of first therapy. The samples were analyzed to determine both the expression of ZAP-70 and the IGHV mutational status. Peripheral-blood mononuclear cells were isolated by density-gradient centrifugation with the use of Ficoll-Paque Plus (Amersham Biosciences). The peripheral blood mononuclear cells (PBMCs) obtained from these patients were composed of >98% leukemic CD5+/CD19+B cells.

RNA Extraction and qRT-PCR

RNA from CLL patients was extracted using standard TRIZOL (Invitrogen, Carlsbad, Calif.) methods. The quality of the RNAs has been analyzed for each sample on the Agilent 2100 Bioanalyzer. RNA integrity number (R.I.N.) was on average 9.2 and on median 9.5 calculated on all samples enrolled in this study. Mature microRNA expression was assayed by Taqman MicroRNA assay (Applied Biosystem) and normalized on RNU44 (P/N: 4373384) according to the manufacturer's protocol. Ten nanograms of total RNA from all samples was reverse transcribed in two days (one day for training set and one day for validation set) in order to avoid technical problems. Each sample was analyzed in triplicate on 384-wells and all the plates were run on the same machine. The level of miRNA was measured using Ct (threshold-cycle). The amount of target, normalized to an endogenous reference is given by: 2^{-ΔCt} (Comparative Ct method, Applied Biosystem).

Cell Culture and Transfection

Cell lines and transfection. HeLa cell lines (all from American Type Culture Collection) were cultured with RPMI-1640 medium with 10% fetal bovine serum. Precursor-hsa-miR-181b precursor (Sanger Accession No. MI0000270) and negative control 2 ribo-oligonucleotide were purchased from Applied Biosystems/Ambion. 2′-O-Me-antisense oligonucleotides (AMO) against miR-181b and against the GFP gene (AMO Negative Control) were purchased from Fidelity Systems. Transfection of miRNAs, AMOs, and expression vectors was carried out with Lipofectamine 2000 (Invitrogen) in accordance with the procedures of the manufacturer.

Luciferase assays and vectors. The human 3′-untranslated region (UTR) of MCL1 was amplified by PCR using the primers MCL1_{—}1212F: 5′-CCGctcgagTAACCAACCACCACCACCAC-3′ [SEQ ID NO:1]; MCL1_{—}3899R: 5′-ATAAGAATgcggccgcCGTTGGTCCTAACCCTTCCTG-3′ [SEQ ID NO:2] and cloned downstream of the firefly luciferase gene psiCHECK-2 Vector (Promega). Substitutions and deletions into the miR-181b binding sites of the MCL1 3′UTR gene was introduced by using Quick-Change site-directed mutagenesis kit following the instructions of the manufacturer and using the primers Mcl1-181 mut F2_{—}5′-TAAGATGACTAAGCCAATGGGGAAGAATTGCCCTG-3′[SEQ ID NO:3]; Mcl1-181 mut R2_{—}5′-CTTCCCCATTGGCTTAGTCATCTTATTCATACC-3′[SEQ ID NO:4].

Transfection was conducted in HeLa cells cultured in 24-well plates, each well was cotransfected with 400 ng of psiCHECK-2 vector and 100 nM of miR-181b or negative control 2 or AMOs, or methylated control oligonucleotide. Twenty-four hours after transfection, firefly and renilla luciferase activities were measured using the Dual-Luciferase Report Assay (Promega).

Western blot analysis. HeLa cell lines were transfected with 100 nmolar of miR-181b, AMOs, and control sequences in 6-well plates. After 48 and 72hrs, cells were collected, lysed in RIPA buffer (Sigma), loaded with Laemmli 5× buffer, and analyzed by Western blot to assess the expression of MCL1 by using monoclonal antibody (antibody sc-819; Santa-Crutz). Detection was conducted by chemiluminescent enhanced assay (WesternBreeze Chemiluminescent Kit; Invitrogen). β-Actin antibody (β-actin antibody no. 4976; Cell Signaling) has been used to normalize the loaded protein amount. Samples from patients were lysed as described above and the expression of TCL1 and BCL2 was assessed by using specific antibodies (sc-32331, Santa Crutz; M0887, Dako). To quantify Western blot signals, digital images of autoradiographies were acquired with Fluor-S Multilmager, and band signals were quantified in the linear range of the scanner using specific densitometric software (Quantity One).

Bioinformatics Analysis

MicroRNA microarray profiling RNA from 2 sequential samples obtained from 23 patients with progressive disease was performed. Average values of the replicate spots of each miRNA were background subtracted, normalized, and further analyzed. Normalization was performed using the quantile method. The inventors selected the miRNAs measured as present in at least as many samples as the smallest class in the data set (50%). Absent calls were threshold to 3.3 (log2 scale) before statistical analysis, representing the average minimum intensity level detectable in the system. More than 95% of blank probes (i.e., negative controls) fell below the threshold value of 3.3. MiRNAs that are differentially expressed in 2 groups were identified using the “Class Comparison among genes” within BRB-Array tools version 3.6.0 developed by Richard Simon and Amy Peng Lam (Simon R, Lam A, Li MC, et al: Analysis of Gene Expression Data Using BRB-Array Tools. Cancer Inform 3:11-7, 2007). The criterion for inclusion of a gene in the gene list is a p-value less than a specified threshold value (0.05).

Statistical Analysis

Two sample t-tests comparing miR-181b values between groups were made on the log-scale where normality and equal variance assumptions did not appear to be violated. Association between starting and ending point were examined with Mann-Whitney' s test. Associations between dichotomous categorical variables, such as the property, IGHV≧98% and ZAP70≧20%, were examined using Fisher's exact test. The time from last time point to initial treatment was estimated by the method of Kaplan-Meier and assessed by the log-rank test. The prognostic value of miR-181b values having a starting value <0.005 or/and a start to end drop of at least 50% (called the property) in determining time to treatment was examined using Cox regression analysis under the proportional hazards model. Here the inventors also examined the possible independent predictive value of ZAP-70 values ≧20% and IGHV values ≧98% while controlling for time on study. All P values were two-sided.

### Example 2

### Dysregulated miRNAs in CLL Patients with Progressive Disease

The characteristics of the patients with progressive and stable disease in the training and the validation sets are shown in Table 1. Progression was defined as a change to a more advanced clinical stage and/or the need for treatment according to the parameters defined in the Workshop on Chronic Lymphocytic Leukemia (Hallek M, Cheson BD, Catovsky D, et al: Guidelines for the diagnosis and treatment of chronic lymphocytic leukemia: a report from the International Workshop on Chronic Lymphocytic Leukemia updating the National Cancer Institute-Working Group 1996 guidelines. Blood 111:5446-56, 2008). In the training set the median time on study was 31 months (IQR 14-41) for patients with a progressive disease and 23 months (IQR 10-42) for those with a stable disease. For the latter group an additional median time of 41 months of follow-up since the last time point was taken into account to verify that the disease remained stable. In the validation set, the median time was 35 months (IQR 16-51) for patients with progressive disease and, for those with a stable disease, 27 months (IQR 15-40) plus an additional median time of follow-up of 46 months.

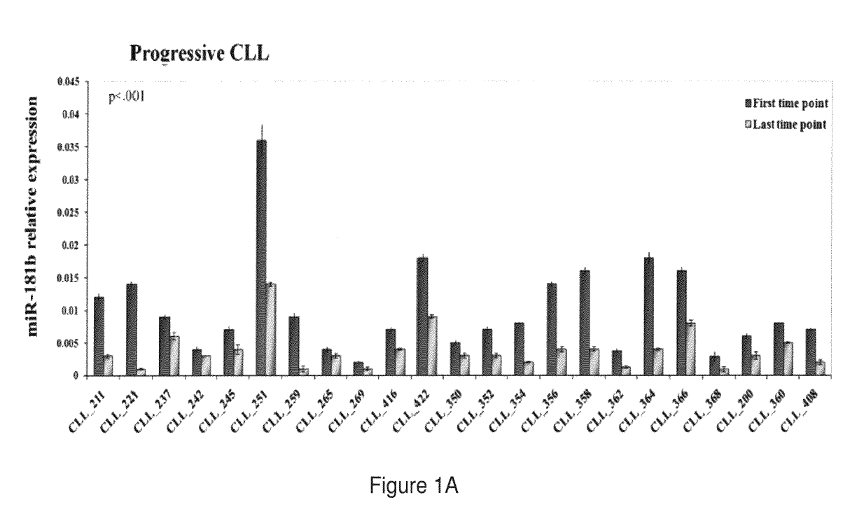

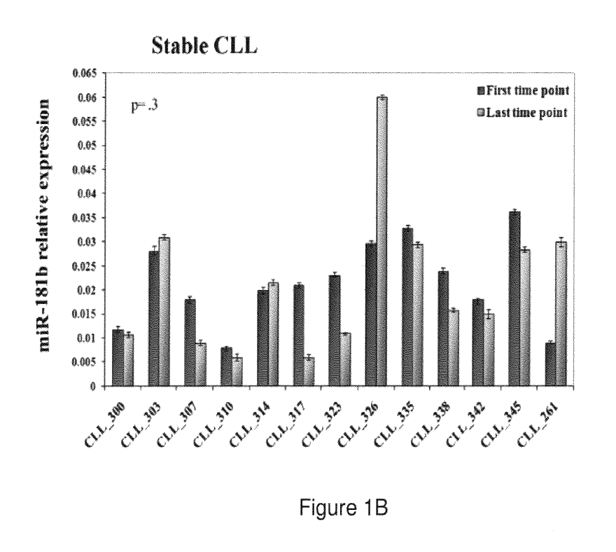

The inventors compared miRNA profiles of 2 sequential samples obtained from 23 patients with a progressive disease in the training cohort. The last time point is a more aggressive form as compared to its previous counterpart (parameters defined according to Hallek et al.). By using the Class Comparison within BRB-Array tools, the inventors identified 15 miRNAs out of 474 human mature miRNA with a P <0.05 but only one with a FDR (false discovery rate) filter <20% (Table 2). MiR-181b was down-regulated in the more aggressive form of the disease compared to its previous counterpart. This result was confirmed by quantitative Real-Time PCR (qRT-PCR) on the total cohort (P<0.001; FIG. 1A).

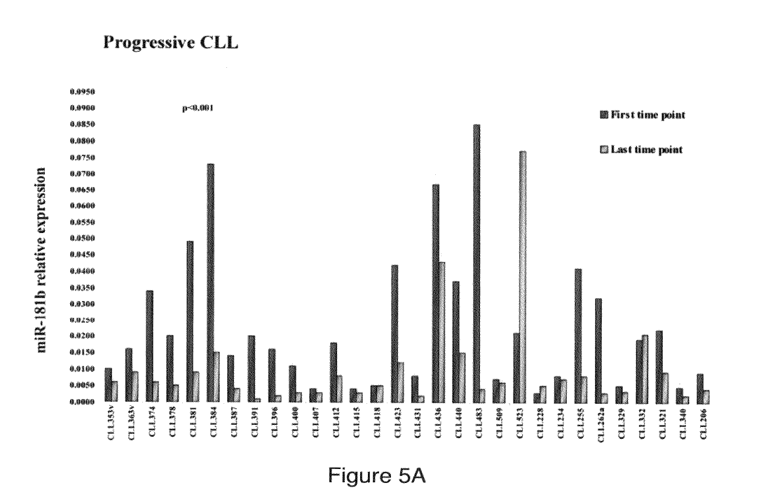

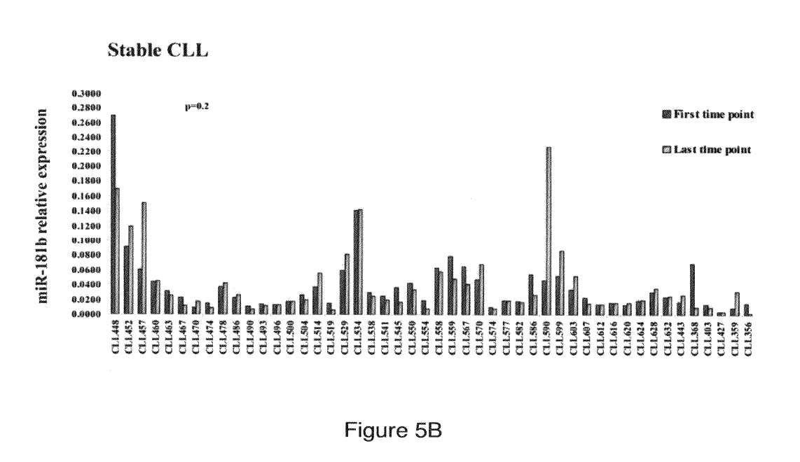

To evaluate the specificity of this finding, the inventors analyzed the expression of miR-181b in 2 sequential samples obtained from 13 patients with the stable course of the disease. The inventors observed that miR-181b values did not undergo significant alteration (P=0.3) between the two time points (FIG. 1B). To validate this data the inventors selected a cohort of 78 patients with either progressive or stable disease in which the criteria for defining the form of the disease matched those of the training set with the only exception of the WBC (white blood cell). The reason for chosen these patients in the validation set without a significant increase of the WBC during the progression was to evaluate if the decrease of the miR-181b occurs independently of this parameter (i.e. WBC). Decrease in miR-181b expression during the progression obtained in the training set was confirmed in the validation cohort (FIG. 5).

TABLE 1
Molecular and clinical features of progressive and stable CLL patients
included in the training and validation set

| | | | | | | ZAP-70 | ZAP-70 |
|---|---|---|---|---|---|---|---|
| | | | Therapy | IGHV | IGHV | positive | positive |
| Samples | | | need | Homology ≧98% | Homology <98% | cells ≧20% | cells <20% |
| Characteristics | FTP^{†} | LTP^{‡} | No. patients | No. patients | No. patients | No. patients | No. patients |
| Training Set | | | | | | | |
| Progressive | | | 18 | 10 | 13 | 8 | 15 |
| (N = 23) | | | | | | | |
| β2m median | 2 | 2.7 | | | | | |
| WBC median | 48.7 | 136.5 | | | | | |
| Stable (N = 13) | | | 0 | 3 | 10 | 3 | 10 |
| β2m median | 1.8 | 1.6 | | | | | |
| WBC median | 26.4 | 30.9 | | | | | |
| Validation Set | | | | | | | |
| Progressive | | | 27 | 18 | 12 | 17 | 13 |
| (N = 30) | | | | | | | |
| β2m median | 1.7 | 2.5 | | | | | |
| WBC median | 24.9 | 31.8 | | | | | |
| Stable (N = 48) | | | 0 | 6 | 42 | 12 | 36 |
| β2m median | 1.8 | 1.9 | | | | | |
| WBC median | 19.4 | 20.2 | | | | | |

^{†}FTP, first time point
^{‡}LTP, last time point

TABLE 2
MiRNAs deregulated between two sequential time points from CLL
patients with a progressive disease.

| p-value^{†} | FDR^{§} | Fold change^{‡} | microRNAs |
|---|---|---|---|
| .0006 | .18 | 0.32 | miR-181b |
| .0015 | .21 | 0.56 | miR-130b |
| .0032 | .29 | 0.35 | miR-126* |
| .0066 | .46 | 0.54 | miR-296-3p |
| .0092 | .47 | 0.65 | miR-223 |
| .0125 | .47 | 0.61 | miR-130a |
| .0128 | .47 | 1.44 | miR-125a-3p |
| .0134 | .47 | 1.22 | miR-668 |
| .0152 | .47 | 0.64 | miR-299-3p |
| .0258 | .64 | 1.46 | miR-499-3p |
| .0274 | .64 | 1.21 | miR-412 |
| .0275 | .64 | 0.63 | miR-192 |
| .0357 | .72 | 1.24 | miR-193b |
| .0358 | .72 | 0.56 | miR-340 |
| .0427 | .80 | 1.36 | miR-93* |

^{†}p-value reported is the results of the class comparison analysis by using the approach “last time point vs first time point”. The analysis was performed by using Biometric Research Branch (BRB) array tool version 3.6.0.
^{§}FDR = False discovery Rate.
^{‡}Fold change is the fold-ratio of the geometric means of microRNA expressions of paired samples with progressive disease. It is calculated by using class comparison within BRB array tool.

### Example 3

### MiR-181b Expression Values Specifically Decrease During the Progression of CLL

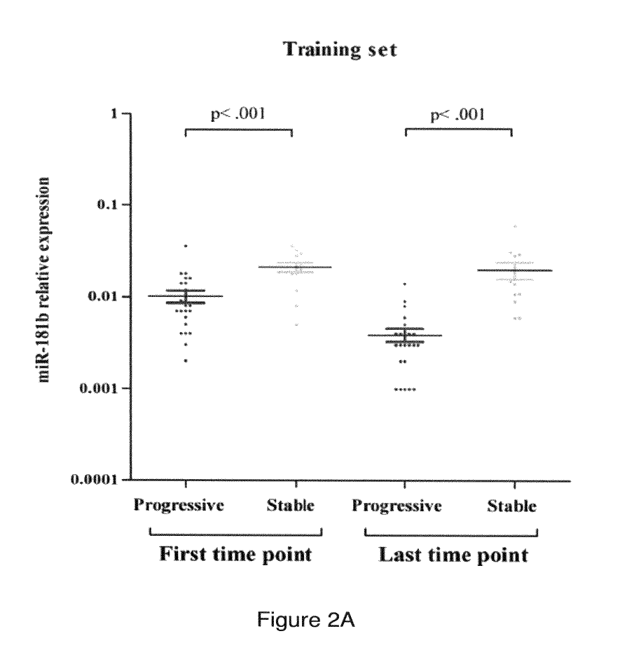

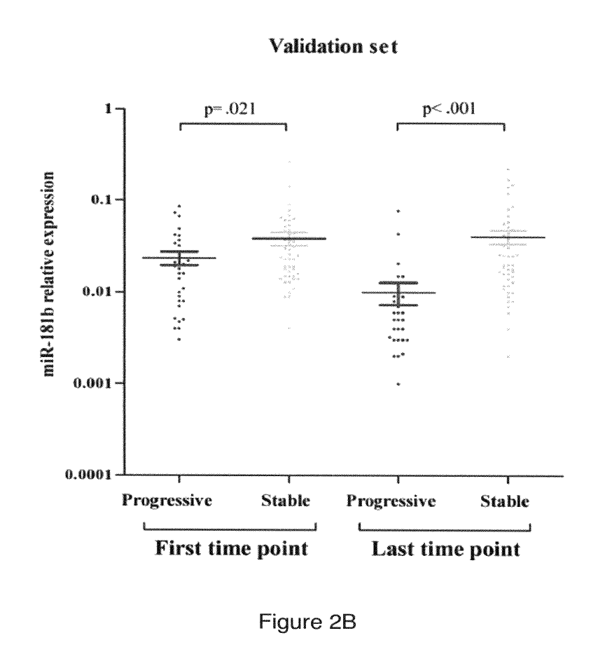

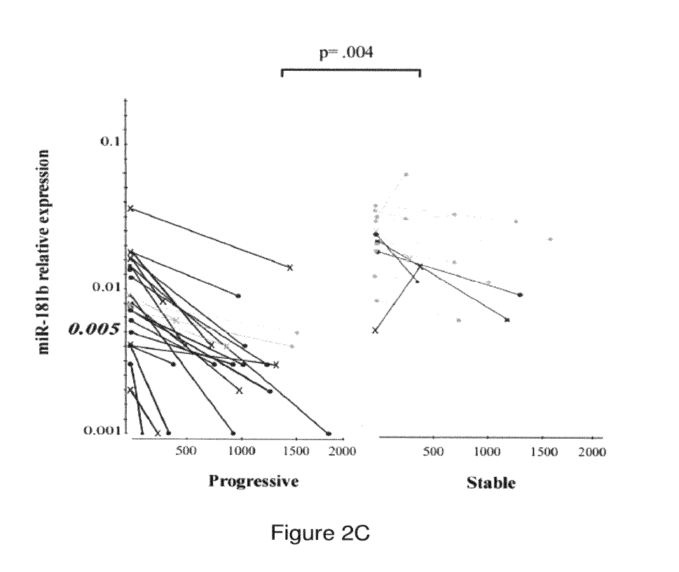

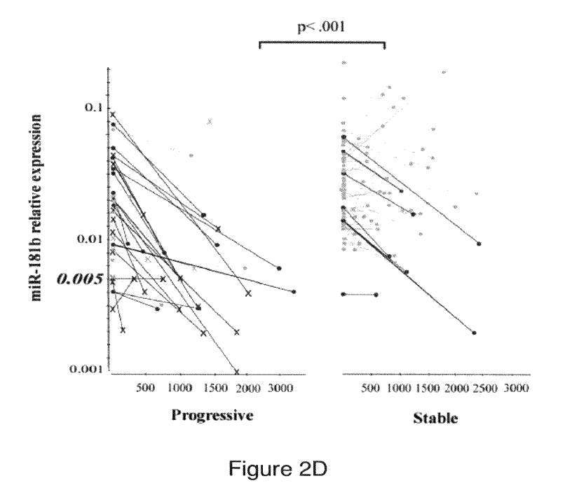

To assess whether the overall expression values of the miR-181b are lower in samples obtained from patients with progressive rather than stable disease, the inventors compared starting and ending values in the 2 subgroups. In the training set, patients that eventually progressed had starting values on average 56% (95% confidence interval, 33% to 72%; P<0.001, Mann-Whitney test) lower than those whose disease remained stable, while the difference between the ending values increased on average to 81% (95% confidence interval, 69 to 89; P<0.001) (FIG. 2A).

With the validation set the inventors confirmed the previous finding in that starting values of samples from patients with progressive CLL are lower than those from patients with a stable disease, 39% average (95% confidence interval, 7% to 60%; P=0.021), whereas between the ending values the difference reached the average of 78% (95% confidence interval, 67% to 86%; P<0.001) (FIG. 2B).

Moreover, to identify parameters that best distinguish progressive from stable disease, the inventors determined the miR-181b expression at all time points. A decrease of 50% or more between the first and the last time point, and a miR-181b value at the starting time point ≦0.005 significantly differentiated the 2 CLL subgroups (P=0.004, training set; P<0.001, validation set; Fisher's exact test) (FIG. 2C and FIG. 2D). The inventors observed that 83% and 73% of the progressive patients, in the training and validation sets respectively, have at least one of these properties, whereas they are present in only 30% and 15% of those with stable disease. A decrease of 50% or more was considered since miR-181b expression undergoes small fluctuation over time, and the value <0.005 was chosen as cutoff based on the training set where fewer stable ( 1/13) had such low miR-181b expression at the starting time point as compared to progressive CLLs ( 6/23). In this analysis the decrease between the first and the last time point was considered. This is a conservative approach since the results would appear stronger by looking at all time points of additional patients with progressive disease (CLL_{—}245, training set: CLL_{—}363v, CLL_{—}436, validation set; Data summarized in Table 4 with a decrease of 50% in miR-181b expression between sequential samples. The inventors further analyzed how well the expression of miR-181b, could discriminate a progressive from a stable disease as compared to the IGHV mutational status and ZAP-70 expression. The inventors found that miR-181b was the most significant biomarker of progression in these cohorts of samples (Table 3).

TABLE 3
Distribution of the subgroup defined on the bases of the properties (drop of the miR-181b
expression values of at least 50% between the first and the last point and/or value ≦0.005 at the
starting point), IGHV mutational status and ZAP-70 expression in progressive and stable CLL.

| | Training Set | | | Validation Set | | |
|---|---|---|---|---|---|---|
| | Progressive | Stable | | Progressive | Stable | |
| | N = 23 | N = 13 | | N = 30 | N - 48 | |
| Subgroup | No | No. | P^{†} | No. | No. | P^{†} |
| With properties | 19 | 4 | .004 | 22 | 7 | <.001 |
| IGHV unmutated | 10 | 3 | .3 | 28 | 6 | <.001 |
| ZAP-positive cells ≧20% | 9 | 3 | .5 | 17 | 12 | .008 |

^{†}P, p-value calculated by Fisher's exact test

### Example 4

### Association Between miR-181b Expression Values and Clinical Outcome in CLL

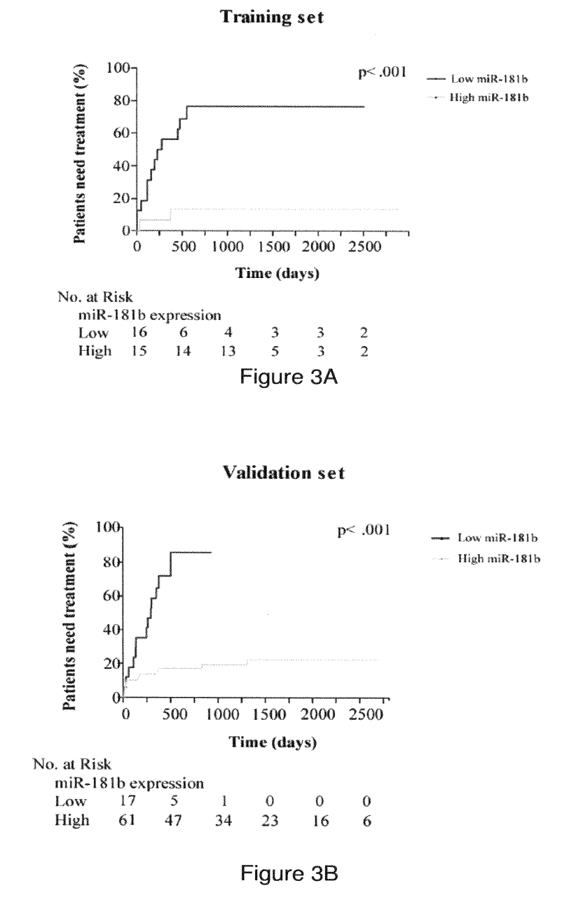

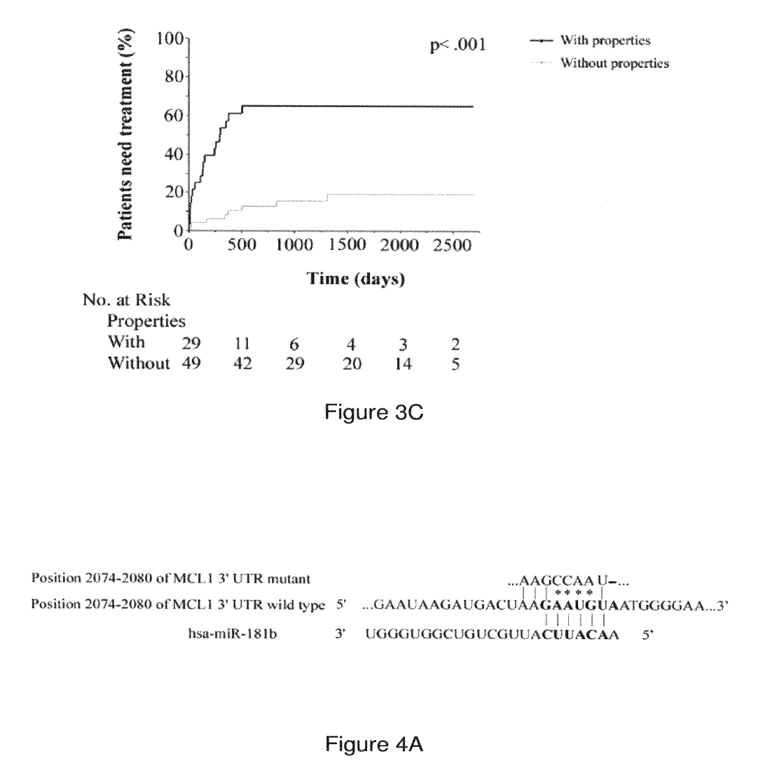

The inventors examined the role of miR-181b expression values as prognostic indicator of time to first treatment. Kaplan-Maier curves (FIG. 3A and FIG. 3B) showed that patients with low expression of miR-181b had a greater risk of needing therapy than those with high levels; the risk was ˜7 times higher (95% Confidence Interval: 2.2-19.4; P<0.001, Log-rank test) in the training set and 19 times higher (95% Confidence Interval: 6.6-55.8; P<0.001) in the validation set. Using the validation cohort the inventors developed a multivariate proportional hazard model to compare the effect of the IGHV mutational status, the ZAP-70 expression and the property previously defined according to the miR-181b expression values on the relative risk of progression. The inventors found a significantly higher hazard of needing treatment in the group with the property (HR, 5.8; 95% confidence interval: 6.6-55.8; P<0.001, FIG. 3C) and for subjects with IGHV≧98% (P˜0.008). After adjusting for these two variables, having ZAP-70≧20% did not significantly affect treatment rate (P˜0.21). Moreover, among 78 patients in the validation cohort the median time to progression was 301 days for the 28 patients with the properties, when compared to the 50 patients lacking these properties where the median was not reached but was already greater than four years.

### Example 5

### Biological Impact of miR-181b in CLL

MiR-181b targets TCL1 and BCL2 genes, which are over-expressed in CLL. Since progression entails an accumulation of leukemic cells due to defects in apoptosis and rapid proliferation.

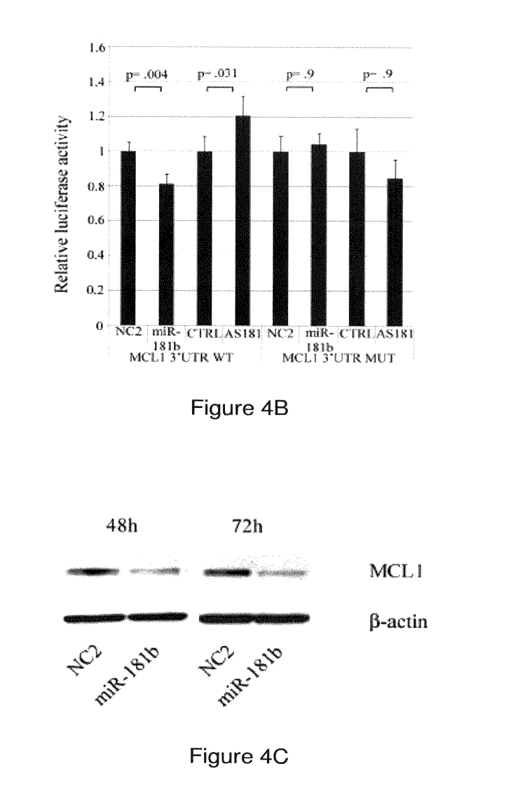

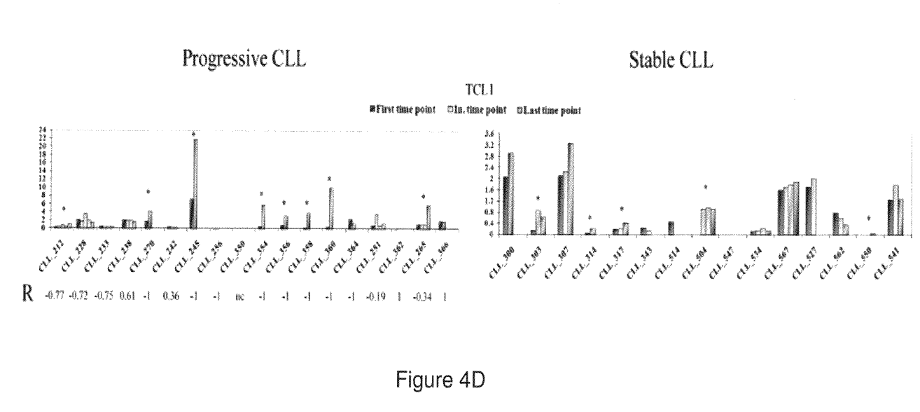

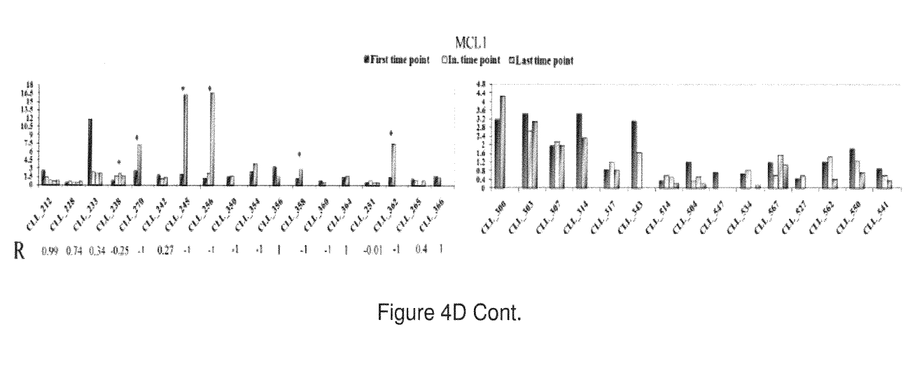

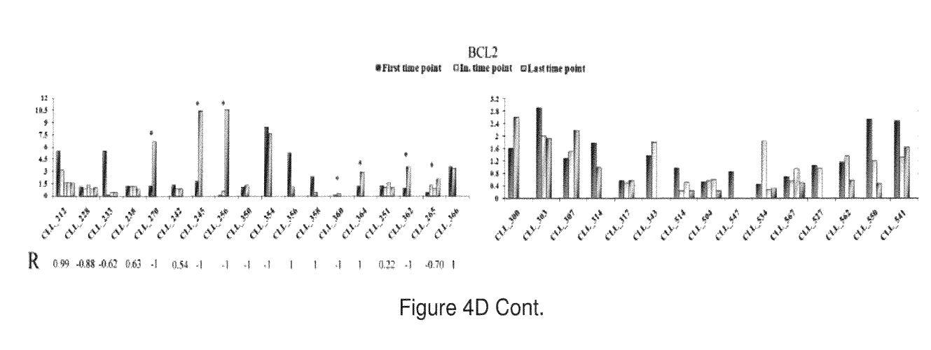

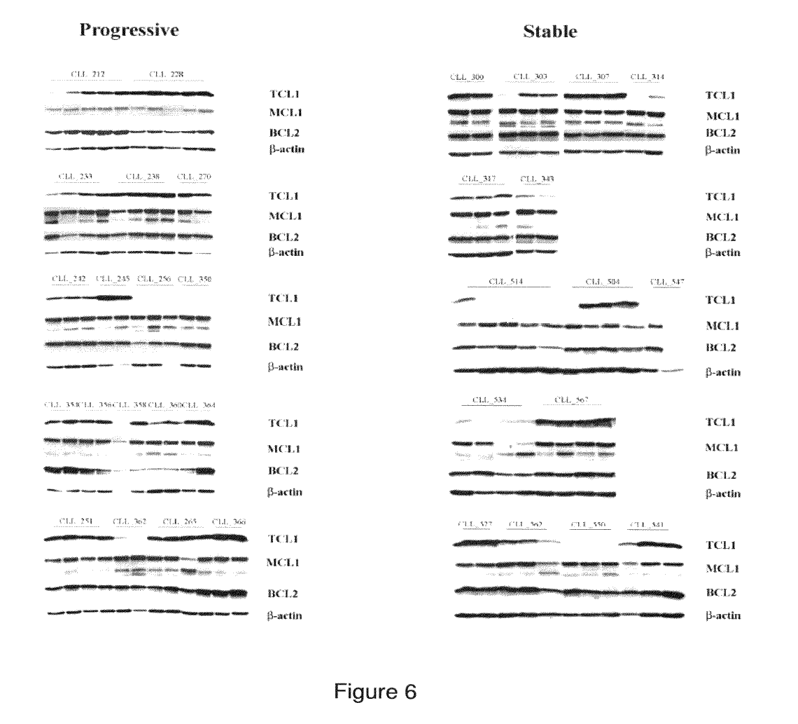

The inventors have now determined that miR-181b regulates the anti-apoptotic factor myeloid cell leukemia sequence 1 (MCL1). Over-expression of this miRNA by oligonucleotide transfection clearly decreased MCL1 protein levels in HeLa cells and inhibited the expression of a reporter vector carrying the MCL1 3′UTR. Mutation of the predicted miRNA binding sites in the reporter vector abrogated this effect, indicating that miR-181b directly interacts with MCL1 3′UTR. Contrarily, transfection of 2′-O-Me-antisense oligonucleotides (AMO) against miR-181b increased the expression of the reporter vector containing MCL1 3′UTR but not that of the vector carrying the mutated sequence (FIG. 4A, FIG. 4B and FIG. 4C). The protein levels of the 3 targets were then analyzed in samples at different time points obtained from patients with either progressive or stable disease. Considering a minimum fold-change of 2 times, protein expression levels of TCL1, MCL1 and BCL2 increased respectively in 5, 0, and 0 out of 15 patients with a stable disease, and in 8, 6 and 7 out of 18 patients with progressive disease over time (P=0.7, TCL1; P=0.02, MCL1; P=0.09, BCL2; Fisher's exact test; FIG. 4D and FIG. 6). However, in 14 patients with progressive disease, at least one of the 3 targets increased its expression over time. In several progressive CLLs, the inventors also observed an inverse correlation between miR-181b and proteins expression (FIG. 4D; R=correlation factor).

### Example 6

### Therapeutic/Prophylactic Methods and Compositions

Also provided herein are methods of treatment and prophylaxis by administration to a subject an effective amount of a therapeutic, i.e., a monoclonal (or polyclonal) antibody, viral vector, Tcl1 mimic or Tcl1 antagonist of the present invention. In a preferred aspect, the therapeutic is substantially purified. The subject is preferably an animal, including but not limited to, animals such as cows, pigs, chickens, etc., and is preferably a mammal, and most preferably human.

Various delivery systems are known and are used to administer a therapeutic of the invention, e.g., encapsulation in liposomes, microparticles, microcapsules, expression by recombinant cells, receptor-mediated endocytosis, construction of a therapeutic nucleic acid as part of a retroviral or other vector, etc. Methods of introduction include, but are not limited to, intradermal, intramuscular, intraperitoneal, intravenous, subcutaneous, intranasal, and oral routes. The compounds are administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers. In one embodiment, administration is by direct injection at the site (or former site) of a malignant tumor or neoplastic or pre-neoplastic tissue.

In a specific embodiment where the therapeutic is a nucleic acid encoding a protein therapeutic the nucleic acid is administered in vivo to promote expression of its encoded protein, by constructing it as part of an appropriate nucleic acid expression vector and administering it so that it becomes intracellular, or coating with lipids or cell-surface receptors or transfecting agents, or by administering it in linkage to a homeobox-like peptide which is known to enter the nucleus. Alternatively, a nucleic acid therapeutic can be introduced intracellularly and incorporated within host cell DNA for expression, by homologous recombination.

The present invention also provides pharmaceutical compositions. Such compositions comprise a therapeutically effective amount of a therapeutic, and a pharmaceutically acceptable carrier or excipient. Such a carrier includes, but is not limited to, saline, buffered saline, dextrose, water, glycerol, ethanol, and combinations thereof. The carrier and composition can be sterile. The formulation will suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition also includes a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it is be dispensed with an infusion bottle containing sterile pharmaceutical grade water or saline. Where the composition is administered by injection, an ampoule of sterile water for injection or saline is provided so that the ingredients are mixed prior to administration.

The therapeutics of the invention is formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of the invention which will be effective in the treatment of a particular disorder or condition will depend on the nature of the disorder or condition, and is determined by standard clinical techniques. In addition, in vitro assays may optionally be employed to help identify optimal dosage ranges. The precise dose to be employed in the formulation will also depend on the route of administration, and the seriousness of the disease or disorder, and is decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

### Example 7

### Method of Treating Cancer Patients

This example describes a method of selecting and treating patients that are likely to have a favorable response to treatments with compositions herein.

A patient diagnosed with cancer ordinarily first undergoes tissue resection with an intent to cure. Tumor samples are obtained from the portion of the tissue removed from the patient. RNA is then isolated from the tissue samples using any appropriate method for extraction of small RNAs that are well known in the art, such as by using TRIZOLTM. Purified RNA is then subjected to RT-PCR using primers specific miR-181b or other differentially expressed miRNAs disclosed, optionally in conjunction with genetic analysis. These assays are run to determine the expression level of the pertinent RNA in the tumor. If differentially expressed miR expression pattern is determined, especially if mutant status is ascertained, the patient is a candidate for treatment with the compositions herein.

Accordingly, the patient is treated with a therapeutically effective amount of the compositions according to methods known in the art. The dose and dosing regimen of the compositions will vary depending on a variety of factors, such as health status of the patient and the stage of the cancer. Typically, treatment is administered in many doses over time.

### Example 8

### Methods of Diagnosing Cancer Patients

In one particular aspect, there is provided herein a method of diagnosing whether a subject has, or is at risk for developing, cancer. The method generally includes measuring the differential miR expression pattern of the miRs compared to control. If a differential miR expression pattern is ascertained, the results are indicative of the subject either having, or being at risk for developing, cancer. In certain embodiments, the level of the at least one gene product is measured using Northern blot analysis. Also, in certain embodiments, the level of the at least one gene product in the test sample is less than the level of the corresponding miR gene product in the control sample, and/or the level of the at least one miR gene product in the test sample is greater than the level of the corresponding miR gene product in the control sample.

### Example 9

### Measuring miR Gene Products

The level of the at least one miR gene product can be measured by reverse transcribing RNA from a test sample obtained from the subject to provide a set of target oligodeoxynucleotides; hybridizing the target oligodeoxynucleotides to a microarray comprising miRNA-specific probe oligonucleotides to provide a hybridization profile for the test sample; and, comparing the test sample hybridization profile to a hybridization profile generated from a control sample. An alteration in the signal of at least one miRNA is indicative of the subject either having, or being at risk for developing, lung cancer, particularly EGFR mutant lung cancer.

### Example 10

### Diagnostic and Therapeutic Applications

In another aspect, there is provided herein are methods of treating a cancer in a subject, where the signal of at least one miRNA, relative to the signal generated from the control sample, is de-regulated (e.g., down-regulated and/or up-regulated).

Also provided herein are methods of diagnosing whether a subject has, or is at risk for developing, a cancer associated with one or more adverse prognostic markers in a subject, by reverse transcribing RNA from a test sample obtained from the subject to provide a set of target oligodeoxynucleotides; hybridizing the target oligodeoxynucleotides to a microarray comprising miRNA-specific probe oligonucleotides to provide a hybridization profile for the test sample; and, comparing the test sample hybridization profile to a hybridization profile generated from a control sample. An alteration in the signal is indicative of the subject either having, or being at risk for developing, the cancer.

### Example 11

### Kits

Any of the compositions described herein may be comprised in a kit. In a non-limiting example, reagents for isolating miRNA, labeling miRNA, and/or evaluating an miRNA population using an array are included in a kit. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. It may also include one or more buffers, such as reaction buffer, labeling buffer, washing buffer, or a hybridization buffer, compounds for preparing the miRNA probes, and components for isolating miRNA. Other kits may include components for making a nucleic acid array comprising oligonucleotides complementary to miRNAs, and thus, may include, for example, a solid support.

For any kit embodiment, including an array, there can be nucleic acid molecules that contain a sequence that is identical or complementary to all or part of any of the sequences herein.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being one preferred solution. Other solutions that may be included in a kit are those solutions involved in isolating and/or enriching miRNA from a mixed sample.

However, the components of the kit may be provided as dried powder(s). When reagents and/or components are provided as a dry powder, the powder can be reconstituted by the addition of a suitable solvent. It is envisioned that the solvent may also be provided in another container means. The kits may also include components that facilitate isolation of the labeled miRNA. It may also include components that preserve or maintain the miRNA or that protect against its degradation. The components may be RNAse-free or protect against RNAses.

Also, the kits can generally comprise, in suitable means, distinct containers for each individual reagent or solution. The kit can also include instructions for employing the kit components as well the use of any other reagent not included in the kit. Instructions may include variations that can be implemented. It is contemplated that such reagents are embodiments of kits of the invention. Also, the kits are not limited to the particular items identified above and may include any reagent used for the manipulation or characterization of miRNA.

It is also contemplated that any embodiment discussed in the context of an miRNA array may be employed more generally in screening or profiling methods or kits of the invention. In other words, any embodiments describing what may be included in a particular array can be practiced in the context of miRNA profiling more generally and need not involve an array per se.

It is also contemplated that any kit, array or other detection technique or tool, or any method can involve profiling for any of these miRNAs. Also, it is contemplated that any embodiment discussed in the context of an miRNA array can be implemented with or without the array format in methods of the invention; in other words, any miRNA in an miRNA array may be screened or evaluated in any method of the invention according to any techniques known to those of skill in the art. The array format is not required for the screening and diagnostic methods to be implemented.

The kits for using miRNA arrays for therapeutic, prognostic, or diagnostic applications and such uses are contemplated by the inventors herein. The kits can include an miRNA array, as well as information regarding a standard or normalized miRNA profile for the miRNAs on the array. Also, in certain embodiments, control RNA or DNA can be included in the kit. The control RNA can be miRNA that can be used as a positive control for labeling and/or array analysis.

The methods and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

### Example 12

### Array Preparation and Screening

Also provided herein are the preparation and use of miRNA arrays, which are ordered macroarrays or microarrays of nucleic acid molecules (probes) that are fully or nearly complementary or identical to a plurality of miRNA molecules or precursor miRNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters.

Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass and silicon. The arrays may vary in a number of different ways, including average probe length, sequence or types of probes, nature of bond between the probe and the array surface, e.g. covalent or non-covalent, and the like. The labeling and screening methods described herein and the arrays are not limited in its utility with respect to any parameter except that the probes detect miRNA; consequently, methods and compositions may be used with a variety of different types of miRNA arrays.

In view of the many possible embodiments to which the principles of the inventors' invention may be applied, it should be recognized that the illustrated embodiments are only preferred examples of the invention and should not be taken as a limitation on the scope of the invention. Rather, the scope of the invention is defined by the following claims. The inventors therefore claim as the inventors' invention all that comes within the scope and spirit of these claims

What is claimed is:

1. A method of identifying poor progression prognosis CLL status in a subject, comprising:
a. comparing the expression level of miR-181b in a first test sample from a subject with CLL and at least one successive test samples from a subject with CLL,
b. identifying poor progression prognosis CLL status in a subject if miR-181b level is decreased from first test sample the at least one successive test sample, or
c. identifying no poor progression prognosis CLL status in a subject if miR-181b level is not decreased from the first test sample the at least one successive test sample.

2. A method of identifying poor progression prognosis CLL status in a subject, comprising:
a. determining the expression level of miR-181b in at least one first test sample;
b. determining the expression level of miR-181b in at least one test sample successive to the first test sample;
c. identifying poor progression prognosis CLL status in a subject if the miR181b level as determined in step b.) is less than miR-181b level as determined in step a.)

3. A method of claim 2, wherein poor progression prognosis CLL status is identified if the miR-181b level as determined in step b.) is at least 20% less than miR-181b level as determined in step a.)

4. A method of claim 2, wherein poor progression prognosis CLL status is identified if the miR-181b level as determined in step b.) is at least 30% less than miR-181b level as determined in step a.).

5. A method of claim 2, wherein poor progression prognosis CLL status is identified if the miR-181b level as determined in step b.) is at least 40% less than miR-181b level as determined in step a.).

6. A method of claim 2, wherein poor progression prognosis CLL status is identified if the miR-181b level as determined in step b.) is at least 50% less than miR-181b level as determined in step a.).

7. A method of claim 2, wherein poor progression prognosis CLL status is identified if the miR-181b level as determined in step b.) is at least 60% less than miR-181b level as determined in step a.).

8. A method of claim 2, wherein step b.) is at least six months after step a.).

9. A method of claim 2, wherein step b.) is at least twelve months after step a.).

10. A method of claim 2, wherein step b.) is at least eighteen months after step a.).

11. A method of claim 2, wherein step b.) is at least twenty-four months after step a.).

12. A method of claim 2, which further comprises identifying clinical stage if poor progression prognosis CLL status is identified.

13. A method of claim 2, which further comprises identifying need for treatment if poor progression prognosis CLL status is identified.

14. A method of claim 2, which further comprises identifying aggressive form of CLL if poor prognosis CLL status is identified.

15. The method of claim 2, wherein a level of expression of miR-181b is assessed by detecting the presence of a transcribed polynucleotide or portion thereof, wherein the transcribed polynucleotide comprises a coding region of miR-181b gene product.

16. The method of claim 2, wherein steps a. and b. are performed in vitro.

17. The method of claim 2, wherein the sample is a CLL-associated body fluid or tissue.

18. The method of claim 2, wherein the sample comprises cells obtained from the patient.

19. A method of identifying poor progression prognosis CLL status in a subject, comprising:
a. comparing the expression level of at least one miR selected from the group consisting of: miR-130b; miR126; miR-296-3p; and miR-223 in a first test sample from a subject with CLL and at least one successive test samples from a subject with CLL,
b. identifying poor progression prognosis CLL status in a subject if the at least one miR expression level is decreased from first test sample the at least one successive test sample, or
c. identifying no poor progression prognosis CLL status in a subject if the miR expression level is not decreased from the first test sample the at least one successive test sample.

20. A method of identifying poor progression prognosis CLL status in a subject, comprising:
a. determining the expression level of at least one miR selected from the group consisting of: miR-130b; miR126; miR-296-3p; and miR-223 in at least one first test sample;
b. determining the expression level of at least one miR selected from the group consisting of: miR-130b; miR126; miR-296-3p; and miR-223 in at least one test sample successive to the first test sample;
c. identifying poor progression prognosis CLL status in a subject if the at least one miR expression level as determined in step b.) is less than the at least one miR expression level as determined in step a.)

21. A method of claim 20, wherein poor progression prognosis CLL status is identified if the miR expression level as determined in step b.) is at least 50% less than miR expression level as determined in step a.).

22. A method of claim 20, wherein step b.) is at least one year after step a.)
